(19) United States
(12) Patent Application Publication
(10) Pub. No.: US 2012/0129187 A1
(43) Pub. Date: May 24, 2012

(54) DIAGNOSTICAL USE OF PEROXIREDOXIN 4

(75) Inventors: Joachim Struck, Berlin (DE); Janina Schulte, Henningsdorf (DE)

(73) Assignee: B.R.A.H.M.S. GMBH, Henningsdorf (DE)

(21) Appl. No.: 13/378,460

(22) PCT Filed: Jun. 15, 2010
(86) PCT No.: PCT/EP2010/058414

§ 371 Date: Jan. 30, 2012

(30) Foreign Application Priority Data
Jun. 16, 2009 (EP) 09162874.7
Aug. 14, 2009 (EP) 09167940.7

(51) Int. Cl. G01N 33/573; C07K 16/18; C12Q 1/28

(52) U.S. Cl. 435/7.4; 435/28; 530/387.9

(57) ABSTRACT

The present invention relates to a method for the diagnosis or prognosis of a disease or clinical condition in a subject comprising the steps of: (i) providing a sample of bodily fluid of a subject, (ii) determining the level of peroxiredoxin 4 (PRX4) or a fragment thereof having at least 20 amino acids residues in length in said sample, and (iii) correlating the level of PRX4 or a fragment thereof with a disease or clinical condition.

### FIELD OF THE INVENTION

The present invention is in the field of clinical diagnostics. Particularly the present invention relates to the determination of the concentration of peroxiredoxin 4 (PRX4) in samples from bodily fluids and the diagnostic use of peroxiredoxin 4.

### BACKGROUND OF THE INVENTION

A major problem for aerobic organisms is exposure to reactive oxygen species (ROS). However, there are numerous biological mechanisms that facilitate removal of ROS within cells. A number of diseases, e.g. cardiac diseases, cancers, infectious diseases and neurodegenerative disorders, have been suggested to be related to imbalances between processes generating ROS on one hand and protective processes on the other hand [Dalle-Donne, I. et al. (2006) Clin. Chem. 52, 601-623, Valko, M. et al. (2007) Int. J. Biochem. Cell Biol. 39, 44-84]. Some ROS also have beneficial effects, e.g. H₂O₂ is an important cytotoxic agent during microbial engulfment by phagocytic immune cells [El-Benna, et al. (2005) Arch. Immunol. Ther. Exp. 53, 199-206]. H₂O₂ can be catalytically generated from NADPH oxidase-derived superoxide anions (O²⁻) in phagocytic immune cells. Furthermore, mammalian cytokines and growth factors are also known to stimulate H₂O₂ production via NADPH oxidases for second messenger signaling purposes [Veal, E. A. et al. (2007) Mol. Cell 26, 1-14; Valko, M. et al. (2007) Int. J. Biochem. Cell Biol. 39, 44-84]. Substantial H₂O₂ is generated as a by-product of metabolic processes such as electron transport ‘leakage’ releasing O²⁻ from the mitochondria [Muller, F. L. (2007) J. Biol. Chem. 279, 49064-49073]. H₂O₂ can directly modify lipids, proteins and nucleic acids. Therefore, effective detoxification pathways exist for the degradation of peroxides. For example, peroxides can be degraded directly by reaction with glutathione, vitamins and other non-enzymatic antioxidants or can be degraded enzymatically, e.g. by catalase (free H₂O₂) or glutathione peroxidases (H₂O₂ and lipid hydroperoxides) [Valko, M. et al. (2007) Int. J. Biochem. Cell Biol. 39, 44-84]. A particular family of peroxidases are the so-called peroxiredoxins which in addition to their peroxidase activity have other functions, such as communicating peroxide stress in the cell.

So far, six peroxiredoxin isoforms have been identified in mammals [Wood, Z. A. et al. (2003) Trends Biochem. Sci. 28, 32-40; Hofman, B. et al. (2002) Biol. Chem. 383, 347-364]. Their cellular locations include the cytosol [PRX1 (Peroxiredoxin 1), 2 and 6], nucleus (PRX 1), mitochondria (PRX 3 and 6) and peroxisomes (PRX 5). All peroxiredoxins comprise a redox-active ‘peroxidatic’ cysteine residue that attacks peroxides. During this process these cysteines are oxidized to cysteine sulfenic acids [Ellis, H. R. (1997) Biochemistry 36, 13349-133567; Choi, H. J. (1998) Nat. Struct. Biol. 5, 400-406; Montemartini, M. (1998) Eur. J. Biochem. 264, 516-524].

Human PRX 1, 2, 3 and 4 in humans contain an additional ‘resolving’ cysteine near their C-terminus and are thus called 2-Cys peroxiredoxins. After peroxide elimination, the peroxidatic cysteine sulfenic acid reacts with the resolving cysteine of its partner to form a stable intermolecular disulfide [Ellis, H. R. (1997) Biochemistry 36, 13349-133567, Hirotsu, S. et al. (1999) Proc. Natl. Acad. Sci. U.S.A. 96, 12333-12338]. For the regeneration of the thiols in the active-site, the disulfide in turn may be reduced by a cell-specific disulfide reductase. 2-Cys peroxiredoxins are typically homodimers, however, they are believed to undergo further fluid transition to toroid decamers and back again [Alphey, M. S. et al. (2000) J. Mol. Biol. 300, 903-916; Schroder, E. et al. (2000) Structure 8, 605-615; Chauhan, R. et al. (2001) Biochem. J. 354, 209-215; Wood, Z. A. et al. (2002) Biochemistry 41, 5493-5504]. Formation of the decamer arranges the active-site for efficient catalysis, whereas Disulfide formation in the active-site destabilizes the complex. Hyperoxidation of the peroxidatic cysteine to a sulfinic acid (SO₂H) derivative is believed to occur at high peroxide concentrations [Rabilloud, T. et al. (2002) J. Biol. Chem. 277, 19396-19401, Wagner, E. et al. (2002) Biochem. J. 366, 777-785] and stabilizes peroxiredoxin decamers by preventing the formation of the resolving disulfide [Schroder, E. et al. (2000) Structure 8, 605-615]. It has been shown in mouse lung cells, that this leads to aggregates of PRX2 decamers whose appearance and subsequent breakdown correlated with arrest and eventual resumption of the cell cycle [Phalen, T. J. (2006) J. Cell Biol. 175, 779-789]. The oligomeric state of PRX 2 can be used as a monitor of cytosolic H₂O₂.

Also other functions of peroxiredoxins have been shown to depend on the oligomeric state, e.g. a chaperone activity associated with high-molecular-mass fractions of Saccharomyces cerevesiae cytosolic peroxiredoxins following stress [Jang, H. H. et al. (2004) Cell 117, 625-635] and with decameric human PRX1 in vitro [Lee, W. et al. (2007) J. Biol. Chem. 282, 22011-22022]. In the case of PRX1, the decamer is covalently stabilized by non-catalytic disulfides preventing dimer-decamer transitions, thereby reducing peroxidase activity and increasing the prevalence of chaperone activity [Lee, W. et al. (2007) J. Biol. Chem. 282, 22011-22022].

PRX4 has an N-terminal sequence, which might be a potential signal for localization into the endoplasmatic reticulum or a membrane or for secretion. PRX4 has been identified a decade ago, however, some confusion exists as to the true role of PRX4 in mammalian cells. Studies show that PRX4 is a cytosolic protein attenuating activity of NF-κB (nuclear factor KB) [Jin, D. Y. et al. (1997) J. Biol. Chem. 272, 30952-30961]. Results from another study indicate that PRX4 is a secretory protein activating NF-κB [Haridas, V. et al. (1998) J. Immunol. 161, 1-6]. This speculation is based on the finding that PRX4 was identified by Western-blot in the culture supernatant of Jurkat cells and HL-60 cells. This, however, does not exclude the possibility that PRX4 originally located in the cytosol simply leaked in the supernatant due to cell damage or necrosis, which has been artificially induced by the cell cultivation process. Other studies showed that rat PRX4 transiently overexpressed in African green monkey cells was translocated and bound at the cell surface [Matsumoto A. et al. (1999) FEBS Lett. 443, 246-250, Okado-Matsumoto, A. et al. (2000) J. Biochem. (Tokyo) 127, 493-501]. The only consistent finding between these studies was the ability of PRX4 to act as a peroxidase in vitro.

Most recent literature teaches that human PRX4 is not a cytosolic protein and not secreted [Tavender, T. J. et al. (2008) Biochem. J. (2008) 411, 191-199]: It traverses into the endoplasmatic reticulum (ER), accompanied by cleavage of the signal sequence, but then, importantly, is retained in the ER and not secreted. In addition, Western-blot analyses of protein precipitates from HT1080, HeLa, HEK-293 cells (human embryonic kidney cells) and HepG2 culture supernatants showed no detectable secretion of PRX IV. The authors conclude that “the finding that PRX4 resides within the human ER clarifies an issue that has remained clouded for the last 10 years.” Apparently consistent with this statement, no evidence whatsoever exists that physiologically or pathophysiologically PRX4 is detectable in the blood circulation. Thus, the question of whether or not PRX4 is secreted or not seemed to be settled.

However, all published experiments addressing the secretability of PRX4 so far were cell line-based, but whether these results reflect the native situation is unclear.

Furthermore, the expression of PRX4 is known to be altered in tissues of certain cancers; see WO 2004/055519 A2. PRX4, a.k.a. NKEF C, is known to be an enhancer of natural killer cells; see U.S. Pat. No. 5,985,612 A1.

Chang et al. (J. Rheumatology 2009; 36(5), 872-80) observed altered concentrations of a large number of proteins, among them PRX4 in samples from synovial tissue in a proteomics approach. In addition, Chang et al. claim that elevated PRX4 concentrations were observed in plasma samples of patients with early rheumatoid arthritis. However, a skilled person would expect the measurement of artefacts under the assay conditions used by Chang et al.

### DESCRIPTION OF THE INVENTION

The present invention is based on the surprising finding of the inventors that PRX4 can be detected in the blood circulation under both physiologic and pathophysiologic conditions. Thus, the present invention pertains to the diagnostic use of PRX4.

The present invention relates to a method for the diagnosis or prognosis of a disease or clinical condition in a subject or for risk stratification or therapy monitoring or therapy guidance in a subject comprising the steps of:
(i) providing a sample of bodily fluid of a subject,
(ii) determining the level of peroxiredoxin 4 (PRX4) or a fragment thereof having at least 20 amino acids residues in length in said sample, and
(iii) correlating the level of PRX4 or a fragment thereof with a disease or clinical condition.

In particular, the invention relates to a method for the diagnosis or prognosis of a disease or clinical condition in a subject comprising the steps of:
(i) providing a sample of bodily fluid of a subject,
(ii) determining the level of peroxiredoxin 4 (PRX4) or a fragment thereof having at least 20 amino acids residues in length in said sample, and
(iii) correlating the level of PRX4 or a fragment thereof with a disease or clinical condition.

More in particular, the invention relates to a method for the diagnosis of a disease or clinical condition in a subject comprising the steps of:
(i) providing a sample of bodily fluid of a subject,
(ii) determining the level of peroxiredoxin 4 (PRX4) or a fragment thereof having at least 20 amino acids residues in length in said sample, and
(iii) correlating the level of PRX4 or a fragment thereof with a disease or clinical condition.

“Diagnosis” in the context of the present invention relates to the recognition and (early) detection of a disease or clinical condition in a subject and may also comprise differential diagnosis. Also the assessment of the severity of a disease or clinical condition may in certain embodiments be encompassed by the term “diagnosis”.

“Prognosis” relates to the prediction of an outcome or a specific risk for a subject suffering from particular disease or clinical condition.

“Risk stratification” in the context of the present invention may relate to the grouping of subjects into different risk groups according to their further prognosis. Risk stratification also relates to stratification for applying preventive and/or therapeutic measures and/or management of patients.

PRX4 or the fragments thereof may be comprised in a homomultimer or in a heteromultimer with other proteins such as other peroxoredoxins or fragments thereof having at least 20 amino acids residues in length. Hence, in the context of the present invention the level of monomers of PRX4 and/or the level of PRX4 comprised in homomultimeric and/or heteromultimeric complexes may be determined. In other words, in a preferred embodiment the PRX4 or the fragment thereof exists in a homomultimeric or heteromultimeric complex and the level of the homomultimeric or heteromultimeric complex is determined.

The amino acid sequence of PRX4 is set forth in SEQ ID NO:1.

“Determining the level of peroxiredoxin 4 (PRX4) or a fragment thereof having at least 20 amino acids residues in length in said sample” relates to the determination of PRX4 or the respective fragments thereof in the sample independent of whether PRX4 and/or the respective fragments are present as monomers or in a multimeric complex, be it a homomultimeric or heteromultimeric complex. In other words the determination of PRX4 or the fragments thereof encompasses the determination of respective homo- or heteromultimers thereof.

A “subject” in the context of the present invention is a human or non-human mammal. For example the subject may be a patient being suspected of having a disease or clinical condition associated with or caused by oxidative stress or being diagnosed with such a disease or clinical condition. Particularly in the latter case the method may be used for diagnosis, differential diagnosis, risk stratification, prognosis, stratification for applying preventive and/or therapeutic measures and/or managements of patients, therapy monitoring, and therapy guidance of a disease or clinical condition.

The term “patient” as used herein refers to a living human or non-human mammal that is receiving medical care or that should receive medical care due to a disease. This includes individuals with no defined illness who are being investigated for signs of pathology. Thus, the methods and assays described herein are applicable to both human and veterinary disease.

The disease or clinical condition diagnosed with the methods of the present invention is preferably associated with oxidative stress. The disease or clinical condition which may be diagnosed according to the present invention may in a particular embodiment be selected from the group consisting of infectious disease, cardiac disease, sepsis (including severe sepsis and septic shock), pancreatitis, diseases of the gastrointestinal tract, cancer, diabetes mellitus, rheumatoid arthritis, kidney disease, and neurodegenerative disorders. In another embodiment the disease or clinical condition is selected from the group consisting of infectious disease, cardiac disease, sepsis, pancreatitis, diseases of the gastrointestinal tract, cancer, diabetes mellitus, kidney disease, and neurodegenerative disorders. In another embodiment the disease or clinical condition is selected from the group consisting of infectious disease, cardiac disease, sepsis, pancreatitis, diseases of the gastrointestinal tract, diabetes mellitus, kidney disease, and neurodegenerative disorders.

In another particular embodiment of the invention, the disease or clinical condition is not rheumatoid arthritis.

Therefore, in this particular embodiment, the present invention relates to a method for the diagnosis or prognosis of a disease or clinical condition in a subject or for risk stratification or therapy monitoring or therapy guidance in a subject comprising the steps of:
(i) providing a sample of bodily fluid of a subject,
(ii) determining the level of peroxiredoxin 4 (PRX4) or a fragment thereof having at least 20 amino acids residues in length in said sample, and
(iii) correlating the level of PRX4 or a fragment thereof with a disease or clinical condition which is not rheumatoid arthritis.

In yet another particular embodiment of the invention, the disease or clinical condition is not a disease or clinical condition selected from the group consisting of rheumatoid arthritis, osteoarthritis and ankylosing spondylitis.

Therefore, in this particular embodiment, the present invention relates to a method for the diagnosis or prognosis of a disease or clinical condition in a subject or for risk stratification or therapy monitoring or therapy guidance in a subject comprising the steps of:
(i) providing a sample of bodily fluid of a subject,
(ii) determining the level of peroxiredoxin 4 (PRX4) or a fragment thereof having at least 20 amino acids residues in length in said sample, and
(iii) correlating the level of PRX4 or a fragment thereof with a disease or clinical condition which is not rheumatoid arthritis, osteoarthritis and ankylosing spondylitis.

Cardiovascular diseases or cardiac diseases may for example be selected from the group of acute coronary syndrome, atherosclerosis, hypertension, stroke and transient ischemic attack. Diseases of the gastrointestinal tract may for example be colitis ulcerosa or Morbus Crohn. Cancer may for example be colon, breast or pancreas cancer. Kidney disease may for example be chronic or acute kidney disease. Neurodegenerative disorders may for example be selected from the group of Alzheimer's disease, mild cognitive disorders and Parkinson's disease.

As outlined above, PRX4 can exist in monomeric or multimeric form, thus, in a particular embodiment of the method according to the level of a homomultimer, particularly a homodecamer or a homopentamer, of PRX4 may be determined. In another particular embodiment the presence or absence or the level of a heteromultimer of PRX4 may be determined. The multimer—be it homo- or heteromultimer—has preferably an apparent molecular weight in the range of from about 158 kDa to about 660 kDa, preferably 330 kDa+/−50 kDa as determined by size exclusion chromatography using a gel filtration column under non-denaturing conditions.

The presence of PRX4 or the fragments thereof may for example be determined by contacting the sample with at least one PRX4 binder. The at least one binder may for example be an antibody. It is preferred that at least one binder is less than 20% cross-reactive with other proteins, particularly other peroxiredoxins such as PRX1, PRX2, PRX3, PRX5 and PRX6. More preferably, at least one binder is less than 2% cross-reactive with other proteins, particularly other peroxiredoxins, e.g. PRX1, PRX2, PRX3, PRX5 and PRX6. In a particular embodiment the at least one binder binds to an epitope contained in positions 1-73 of PRX4 according to SEQ ID NO:1. In another particular embodiment the at least one binder binds to an epitope contained in positions 39-65 of PRX4 according to SEQ ID NO:1.

To minimize the possibility of obtaining anti-PRX4 antibodies, which exhibit crossreactivity to other members of the Prx familiy, a region was selectedin the N-terminal part of PRX4 as a source of immunogens, for which a corresponding region does not exist in several other members of the Prx family (Prx 1 and 2) (FIG. 1). Thereby, crossreactivity against Prx 1 and 2 was excluded already by design. Only PRX3 has a region that extends N-terminally Prx 1 and 2 and thus could potentially correspond to the N-terminal part of PRX4 (FIG. 1). Both corresponding sequences are highlighted below. The degree of homology between these two sequences is negligible. Thus by developing antibodies against the PRX4 sequence it must be expected already due to this very low sequence homology that these antibodies will not crossreact with PRX3. Experimentally crossreactivity could be assessed by quantifying the binding of an antibody to be tested against equimolar amounts of the respective chemically synthesized peptides representing the N-terminal moieties of PRX4 and PRX3 respectively. Precisely, the antibody to be tested would be labeled as described. Peptides would be synthesized as described. Peptides would be coated as described for “Coating of antibodies”. Binding of a labeled antibody would be performed on peptide-coated tubes as described under “Immunoassay A.2” only by omitting sample. Crossreactivity (%) would be calculated by dividing the amount (signal) of antibody bound to the PRX3 peptide through the amount (signal) of antibody bound to the PRX4 peptide.

### PRX4

```
SEQ ID NO: 8
Met Glu Ala Leu Pro Leu Leu Ala Ala Thr Thr Pro Asp His Gly Arg
1 5 10 15
His Arg Arg Leu Leu Leu Leu Pro Leu Leu Leu Phe Leu Leu Pro Ala
            20 25 30
Gly Ala Val Gln Gly Trp Glu Thr Glu Glu Arg Pro Arg Thr Arg Glu
        35 40 45
Glu Glu Cys His Phe Tyr Ala Gly Gly Gln Val Tyr Pro Gly Glu Ala
    50 55 60
Ser Arg Val Ser Val Ala Asp His Ser Leu His Leu Ser Lys Ala Lys
65 70 75 80
Ile Ser Lys Pro Ala Pro Tyr Trp Glu Gly Thr Ala Val Ile Asp Gly
                    85 90 95
Glu Phe Lys Glu Leu Lys Leu Thr Asp Tyr Arg Gly Lys Tyr Leu Val
              100 105 110
Phe Phe Phe Tyr Pro Leu Asp Phe Thr Phe Val Cys Pro Thr Glu Ile
        115 120 125
Ile Ala Phe Gly Asp Arg Leu Glu Glu Phe Arg Ser Ile Asn Thr Glu
      130 135 140
Val Val Ala Cys Ser Val Asp Ser Gln Phe Thr His Leu Ala Trp Ile
145 150 155 160
Asn Thr Pro Arg Arg Gln Gly Gly Leu Gly Pro Ile Arg Ile Pro Leu
                  165 170 175
Leu Ser Asp Leu Thr His Gln Ile Ser Lys Asp Tyr Gly Val Tyr Leu
              180 185 190
Glu Asp Ser Gly His Thr Leu Arg Gly Leu Phe Ile Ile Asp Asp Lys
        195 200 205
Gly Ile Leu Arg Gln Ile Thr Leu Asn Asp Leu Pro Val Gly Arg Ser
    210 215 220
Val Asp Glu Thr Leu Arg Leu Val Gln Ala Phe Gln Tyr Thr Asp Lys
225 230 235 240
His Gly Glu Val Cys Pro Ala Gly Trp Lys Pro Gly Ser Glu Thr Ile
                  245 250 255
Ile Pro Asp Pro Ala Gly Lys Leu Lys Tyr Phe Asp Lys Leu Asn
                260 265 270
```

### PRX3

```
SEQ ID NO: 9
Met Ala Ala Ala Val Gly Arg Leu Leu Arg Ala Ser Val Ala Arg His
1 5 10 15
Val Ser Ala Ile Pro Trp Gly Ile Ser Ala Thr Ala Ala Leu Arg Pro
              20 25 30
Ala Ala Cys Gly Arg Thr Ser Leu Thr Asn Leu Leu Cys Ser Gly Ser
        35 40 45
Ser Gln Ala Lys Leu Phe Ser Thr Ser Ser Ser Cys His Ala Pro Ala
    50 55 60
Val Thr Gln His Ala Pro Tyr Phe Lys Gly Thr Ala Val Val Asn Gly
65 70 75 80
Glu Phe Lys Asp Leu Ser Leu Asp Asp Phe Lys Gly Lys Tyr Leu Val
                  85 90 95
Leu Phe Phe Tyr Pro Leu Asp Phe Thr Phe Val Cys Pro Thr Glu Ile
              100 105 110
Val Ala Phe Ser Asp Lys Ala Asn Glu Phe His Asp Val Asn Cys Glu
        115 120 125
Val Val Ala Val Ser Val Asp Ser His Phe Ser His Leu Ala Trp Ile
    130 135 140
Asn Thr Pro Arg Lys Asn Gly Gly Leu Gly His Met Asn Ile Ala Leu
145 150 155 160
Leu Ser Asp Leu Thr Lys Gln Ile Ser Arg Asp Tyr Gly Val Leu Leu
                  165 170 175
Glu Gly Ser Gly Leu Ala Leu Arg Gly Leu Phe Ile Ile Asp Pro Asn
              180 185 190
Gly Val Ile Lys His Leu Ser Val Asn Asp Leu Pro Val Gly Arg Ser
        195 200 205
Val Glu Glu Thr Leu Arg Leu Val Lys Ala Phe Gln Tyr Val Glu Thr
    210 215 220
His Gly Glu Val Cys Pro Ala Asn Trp Thr Pro Asp Ser Pro Thr Ile
225 230 235 240
Lys Pro Ser Pro Ala Ala Ser Lys Glu Tyr Phe Gln Lys Val Asn Gln
                  245 250 255
```

PRX4 and/or fragments thereof can for example be determined in an immuno assay, preferably a sandwich assay. In a particular embodiment such a sandwich assay comprises at least two binders which can bind the same epitope or overlapping epitopes of PRX4. In this case this epitope or these epitopes of the binders are preferably contained in positions 39-65 of PRX4 according to SEQ ID NO:1.

In another preferred embodiment the sandwich assay comprises at least two binders which can bind different epitopes of PRX4. Preferably, one binder binds to an epitope contained in positions 39-65 of PRX4 according to SEQ ID NO:1 and a second binder binds to an epitope contained in positions 51-65 of PRX4 according to SEQ ID NO:1.

Sandwich immuno assays can for example be designed as one-step assays or as a two-step assays.

In the methods according to the present invention, prior to or during the determination of PRX4 the sample may be contacted with an agent that leads to an improvement of the ex vivo stability of PRX4 and/or stabile fragments thereof regarding its determination and/or to an improvement of the analytical detection limit of the assay and/or other measures related to the analytical detection limit such as functional assay sensitivity, signal to noise ratio.

“Improvement of the ex vivo stability” means that the immunoreactivity is preferably constant and does not significantly increase or decrease until detection. Said agent may preferably be a reducing agent, such as dithiothreitol (DTT), β-mercaptoethanol, ascorbic acid, or Cu²⁺ ions. DTT is preferred. In this case the final concentration of DTT in the sample preferably is between 1 and 10 mM.

The sample of bodily fluid is preferably selected from the group consisting of a blood sample, a serum sample, a plasma sample, a cerebrospinal fluid sample, a saliva sample, a solubilised tissue sample and an urine sample or an extract of any of the aforementioned samples. It is preferred that the sample is not derived from synovial tissue. Preferably the sample is a serum sample or a plasma sample. Most preferably the sample is a serum sample for all diseases to be determined.

In one very particular embodiment though, the sample is a serum sample and the disease or clinical condition is selected from the group consisting of rheumatoid arthritis, osteoarthritis and ankylosing spondylitis.

In another very particular embodiment the disease or clinical condition is not rheumatoid arthritis and the sample is a serum or plasma sample.

It is preferred that the plasma- or serum sample has been obtained in a way, by which blood cells potentially containing PRX4 are quantitatively separated from plasma or serum. Haemolysis of the blood samples should be avoided in the context of the present invention.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood) for at least 15 minutes at 2000 to 3000 g.

Therefore, it is preferred that plasma samples employed in the context of the present invention have been subjected to centrifugation at more than 1500 g for 30 min, preferably at least at to 2000 g for at least 30 min, more preferably at least at 3000 g for at least 20 min, most preferably at least at 3000 g for at least 30 min.

In one particular embodiment of the invention the plasma sample is not a citrate-treated plasma sample.

“Serum” in the context of the present invention is the undiluted, extracellular portion of blood after adequate coagulation is completed. Coagulation is usually completed after 30 min. Serum can be obtained by centrifugation of the coagulated sample for at least 10 minutes at a minimum speed of 1500 g.

Therefore, it is preferred that serum samples employed in the context of the present invention have been subjected to centrifugation at least at 1500 g for at least 10 min, preferably for at least 15 min, more preferably for at least 20 min. Most preferably the serum sample has been subjected to a centrifugation at least at 3000 g for at least 20 min.

When separating serum or plasma, the temperature should not drop below 15° C. or exceed 24° C.

In the context of the methods and assays of the present invention, in addition to the determination of PRX4 in the sample of the subject the determination of other markers or clinical or laboratory parameters may be performed and accounted for in the correlation with a disease or clinical condition. This means that additional information may be included into the diagnosis, prognosis, risk stratification, therapy monitoring or therapy guidance. Laboratory parameters are for example the levels of other indicative markers in the sample, e.g. peptide markers.

This does not imply, albeit not exclude, that such determinations are technically combined. The use of the additional information may be performed in any kind of mathematical combination of parameters—be it laboratory and/or clinical parameters—that yield a diagnosis, prognosis, risk stratification, therapy monitoring or therapy guidance of the subject. One example of such mathematical combination is the Cox proportional hazards analysis, from which a subject's risk to experience a certain outcome can be derived, but other methods maybe used as well.

The invention also involves comparing the level of PRX4 for the individual with a predetermined value. The predetermined value can take a variety of forms. It can be single cut-off value, such as for instance a median or mean or the 75^{th}, 90^{th}, 95^{th} or 99^{th} percentile of a population. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quartiles, the lowest quartile being individuals with the lowest risk and the highest quartile being individuals with the highest risk.

The predetermined value can vary among particular populations selected, depending on their habits, ethnicity, genetics etc. For example, an apparently healthy, non-smoker population (no detectable disease and no prior history of a disease related to oxidative stress) might have a different ‘normal’ range of markers than a smoking population or a population the members of which have a history of disease related to oxidative stress. Accordingly, the predetermined values selected may take into account the category in which an individual falls. Appropriate ranges and categories can be selected with no more than routine experimentation by those of ordinary skill in the art.

The level of PRX4 and other markers can be obtained by any art recognized method. The level can be determined by immunoassays or other conventional techniques for determining the level of the marker. Recognized methods include sending samples of a patient's body fluid to a commercial laboratory for measurement, but also performing the measurement at the point-of-care.

Suitable markers include but are not restricted to biomarkers such as peptide hormones or fragments thereof or precursors or fragments of precursors of peptide hormones.

Alternatively or additionally, determined levels of PRX4 or fragments thereof maybe combined with clinical parameters. Thus, in another preferred embodiment of the invention the diagnosis, prognosis, risk stratification, therapy monitoring or therapy guidance for the subject is improved by determining and using clinical parameters, in addition to PRX4, selected from the group, but not restricted to these: age, gender, systolic blood pressure, diastolic blood pressure, antihypertensive treatment, body mass index, presence of diabetes mellitus, current smoking.

The levels, i.e. the concentrations, of PRX4 and optionally one or more additional other marker peptides (or fragments thereof or precursors or fragments thereof) in the sample of the patient may for example be attributed to the diagnosis of a patient or prognosis of an outcome, assessing the risk for the patient, differential diagnosis, risk stratification, stratification for applying preventive and/or therapeutic measures and/or managements of patients, therapy monitoring, and therapy guidance of a disease or clinical condition. Particularly, concentrations of PRX4 above a certain threshold value may be indicative for a particular outcome or prognosis or differential diagnosis for a patient.

The levels of the markers including PRX4 as obtained by the methods or the use of the assays according to the present invention may be analyzed in a number of fashions well known to a person skilled in the art. For example, each assay result obtained may be compared to a “normal” value, or a value indicating a particular diagnosis, prognosis or outcome. A particular diagnosis/prognosis may depend upon the comparison of each assay result to such a value, which may be referred to as a diagnostic or prognostic “threshold”. In certain embodiments, assays for one or more diagnostic or prognostic indicators are correlated to a condition or disease by merely the presence or absence of the indicator(s) in the assay. For example, an assay can be designed so that a positive signal only occurs above a particular threshold concentration of interest, and below which concentration the assay provides no signal above background.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy) and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art. See, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In certain embodiments, particular thresholds for one or more markers in a panel are not relied upon to determine if a profile of marker levels obtained from a subject are indicative of a particular diagnosis/prognosis. Rather, the present invention may utilize an evaluation of a marker panel “profile” as a unitary whole. A particular “fingerprint” pattern of changes in such a panel of markers may, in effect, act as a specific diagnostic or prognostic indicator. As discussed herein, that pattern of changes may be obtained from a single sample, or from temporal changes in one or more members of the panel (or a panel response value). A panel herein refers to a set of markers.

As described herein after, a panel response value is preferably determined by plotting ROC curves for the sensitivity (i.e. true positives) of a particular panel of markers versus 1-(specificity) (i.e. false positives) for the panel at various cut-offs. In these methods, a profile of marker measurements from a subject is considered together to provide a global probability (expressed either as a numeric score or as a percentage risk) of a diagnosis or prognosis. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In certain embodiments, marker panels comprising PRX4 are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, marker panels including PRX4 are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/5% of a given measurement.

The skilled artisan will understand that associating a diagnostic or prognostic indicator, with a diagnosis or with a prognostic risk of a future clinical outcome is a statistical analysis. For example, a marker level of greater than X may signal that a patient is more likely to suffer from an adverse outcome than patients with a level less than or equal to X, as determined by a level of statistical significance. Additionally, a change in marker concentration from baseline levels may be reflective of patient prognosis, and the degree of change in marker level may be related to the severity of an outcome. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In yet other embodiments, multiple determinations of PRX4 and optionally further markers can be made, and a temporal change in the marker can be used to determine a diagnosis or prognosis of a disease or clinical condition or for risk stratification or therapy monitoring or therapy guidance in a subject suffering from a disease or clinical condition. For example, a PRX4 level in a subject sample may be determined at an initial time, and again at a second time from a second subject sample. In such embodiments, an increase in the level from the initial time to the second time may be indicative of a particular diagnosis, or a particular prognosis. Likewise, a decrease in the level from the initial time to the second time may be indicative of a particular diagnosis, or a particular prognosis.

The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

The term “correlating,” as used herein in reference to the use of diagnostic and prognostic markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis or prognosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a panel of marker levels is correlated to a global probability or a particular outcome.

Suitable threshold levels for the stratification of subjects into different groups (categories) have to be determined for each particular combination of PRX4 level, further markers and/or parameters, medication and disease. This can e.g. be done by grouping a reference population of patients according to their level of PRX4 into certain quantiles, e.g. quartiles, quintiles or even according to suitable percentiles. For each of the quantiles or groups above and below certain percentiles, hazard ratios can be calculated comparing the risk for an adverse outcome, i.e. an “unfavourable effect”, e.g. in terms of survival rate, between those patients who have received a certain medication and those who did not. In such a scenario, a hazard ratio (HR) above 1 indicates a higher risk for an adverse outcome for the patients who have received a treatment than for patients who did not. A HR below 1 indicates beneficial effects of a certain treatment in the group of patients. A HR around 1 (e.g. +/−0.1) indicates no elevated risk but also no benefit from medication for the particular group of patients. By comparison of the HR between certain quantiles of patients with each other and with the HR of the overall population of patients, it is possible to identify those quantiles of patients who have an elevated risk and those who benefit from medication and thereby stratify subjects according to the present invention.

Determining (or measuring or detecting) the level of PRX4 herein may be performed using a detection method and/or a diagnostic assay as explained below.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules (also termed “binders” herein) and target molecules or molecules of interest, the affinity constant is preferably greater than 10⁸ M⁻¹.

In the context of the present invention, “capture molecules” are molecules which may be used to bind target molecules or molecules of interest, i.e. analytes (i.e. in the context of the present invention PRX4 and optionally other markers), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, capture molecules may for instance be selected from the group comprising a nucleic acid molecule, a carbohydrate molecule, a RNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof, preferably a length of at least 20 amino acids.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

The assays can be homogenous or heterogeneous assays, competitive and non-competitive sandwich assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person. (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134), incorporated herein by reference).

In a particularly preferred embodiment the assay comprises two capture molecules, preferably antibodies which are both present as dispersions in a liquid reaction mixture, wherein a first labeling component is attached to the first capture molecule, wherein said first labeling component is part of a labeling system based on fluorescence- or chemiluminescence-quenching or amplification, and a second labeling component of said marking system is attached to the second capture molecule, so that upon binding of both capture molecules to the analyte a measurable signal is generated that allows for the detection of the formed sandwich complexes in the solution comprising the sample.

Even more preferred, said labeling system comprises rare earth cryptates or rare earth chelates in combination with a fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, auch as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in Kirk-Othmer, Encyclopedia of chemical technology, 4^{th} ed., executive editor, J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters.

The present invention also relates to an antibody that binds to an epitope contained in positions 1-73 of PRX4 according to SEQ ID NO:1 and is less than 20% cross-reactive with PRX4-related proteins. Preferably, the antibody is less than 2% cross-reactive with PRX4-related proteins.

In a particular embodiment the antibody binds to an epitope contained in positions 39 to 65 of PRX4 according to SEQ ID NO: 1. In a preferred embodiment the antibody binds to an epitope contained in positions 51 to 65 of PRX4 according to SEQ ID NO:1. In yet another preferred embodiment the antibody binds to an epitope contained in positions 39 to 65 of PRX4 according to SEQ ID NO:1.

In a preferred embodiment the antibody is a monoclonal antibody. Alternatively, the antibody is a polyclonal antibody.

The present invention also pertains to a diagnostic kit comprising at least one antibody according to the invention.

Furthermore, the present invention relates to the use of an organ- or tissue extract and/or enriched or purified fractions thereof containing PRX4 and/or a fragment thereof having at least 20 amino acids residues in length as a source for providing calibrators and/or control samples used in a the determination of PRX4 and/or a and/or a fragment thereof having at least 20 amino acids residues in length. As described herein above, PRX4 or the fragment thereof may be monomeric or exist in a heteromultimer or a homomultimer.

The organ extract may be an extract of any organ containing PRX4. However, it is preferred that the organ extract is a liver extract. The organ is preferably a non-human organ, e.g. a porcine or a bovine organ.

The present invention also relates to the use of the method, the antibody and the kit according to the invention for the diagnosis, differential diagnosis, risk stratification, prognosis, stratification for applying preventive and/or therapeutic measures and/or managements of patients, therapy monitoring, and or therapy guidance of a disease or clinical condition associated with oxidative stress such as infectious disease, cardiac disease, sepsis (including severe sepsis and septic shock), pancreatitis, diseases of the gastrointestinal tract, cancer, diabetes mellitus, rheumatoid arthritis, kidney disease, or neurodegenerative disorders.

### Amino Acid Sequence of Human Peroxiredoxin 4 (PRX4) SEQ ID NO:1

```
                10 20 30 40 50 60
MEALPLLAAT TPDHGRHRRL LLLPLLLFLL PAGAVQGWET EERPRTREEE CHFYAGGQVY
                70 80 90 100 110 120
PGEASRVSVA DHSLHLSKAK ISKPAPYWEG TAVIDGEFKE LKLTDYRGKY LVFFFYPLDF
              130 140 150 160 170 180
TFVCPTEIIA FGDRLEEFRS INTEVVACSV DSQFTHLAWI NTPRRQGGLG PIRIPLLSDL
              190 200 210 220 230 240
THQISKDYGV YLEDSGHTLR GLFIIDDKGI LRQITLNDLP VGRSVDETLR LVQAFQYTDK
              250 260 270
HGEVCPAGWK PGSETIIPDP AGKLKYFDKL N
```

### DESCRIPTION OF DRAWINGS

FIG. 1: Sequence alignment of members of the human peroxiredoxin protein family. The sequence alignment was performed using the BLAST program of www.uniprot.org.

FIG. 2: Dose response curves for PRX4 immunoreactivity. In both panels, assay A.1 was used. Dilutions of porcine liver extract were measured in panel A, and dilutions of peptide PEE27 were measured in panel B.

FIG. 3: Correlation of PRX4 immunoreactivity detected in human sera using a homologous sandwich assay and a heterologous sandwich assay. Assays A.1 (X-axis) and B (Y-axis) were used.

FIG. 4: PRX4 immunoreactivity profiles of a human serum pool (A) or porcine liver extract (B) fractionated by size exclusion HPLC. Elution times of size calibrators are indicated.

FIG. 5: Effect of DTT on detectable PRX4 immunoreactivity. Panel A shows dose-response curves for dilutions of porcine liver extract dependent of the DTT concentrations used in the assay buffer in the first incubation step of assay A.1. Panel B shows a correlation of PRX4 immunoreactivities detected in human serum samples using assay A.1, which either contained or did not contain 3 mM DTT in the first incubation step.

FIG. 6: Effect of DTT on the ex vivo stability of detectable PRX4 immunoreactivity. Shown are mean values obtained for 5 samples.

FIG. 7: PRX4 immunoreactivity measured in clinical samples. Samples are grouped according to the type of disease of the respective patients. Median values for each group are indicated.

FIG. 8: Correlation of the level of PRX4 with the levels of Procalcitonin (PCT) in samples from patients with sepsis, severe sepsis and septic shock (Spearman r=0.33). Levels of PCT are markers for sepsis, severe sepsis and septic shock.

FIG. 9: Comparison of PRX4 recovery from serum and plasma samples after centrifugation at 1500 g, 2000 g, 2500 g and 3000 g. Values have been normalized to values at 3000 g (=100%).

FIG. 10: PRX in patients with sepsis on consecutive days after admission.

FIG. 11: PRX in patients with severe acute pancreatitis.

FIG. 12: PRX in patients with stroke.

### EXAMPLES

### Example 1

### Analysis of Clinical Samples

### Material and Methods

### Peptides

From the known amino acid sequence of human Peroxiredoxin 4 (see SEQ ID NO:1) two regions were selected, which were chemically synthesized by standard procedures (JPT GmbH, Berlin, Germany). These peptides were: PEC13 (SEQ ID NO:2: sequence: ETEERPRTREEEC, i.e. residues 39-51 of PRX4 and SEQ ID NO:1), PCS15 (SEQ ID NO:3: sequence: CHFYAGGQVYPGEAS, i.e. residues 51-65 of PRX4 and SEQ ID NO:1) and PEE27 (SEQ ID NO:4: sequence: ETEERPRTREEEGGGETEERPRTREEE, i.e. residues 39-50 of SEQ ID NO:1 followed by GGG, followed by residues 39-50 of SEQ ID NO:1).

### Monoclonal Antibodies

Monoclonal antibodies directed against PEC13 and PCS15 were generated by standard procedures (Harlow E, Lane D. Antibodies—A Laboratory Manual. Cold Spring Harbor: Cold Spring Harbor Laboratory, 1988; Lane R D. A short-duration polyethylene glycol fusion technique for increasing production of monoclonal antibody-secreting hybridomas J Immunol Methods 1985; 81:223-8.) Briefly, peptides were conjugated to BSA by using Sulfo-MBS (m-maleimidobenzoyl-N-hydroxysuccinimid ester). With these conjugates Balb/c mice were immunized and boostered, and spleen cells were fused with SP2/0 myeloma cells to generate hybridoma cell lines. Cell lines were screened for their ability to secrete antibodies that would bind to the immunogenic peptides, which were coated on a solid polystyrene phase. With this approach, cell lines secreting monoclonal antibodies 340/4F2 and 340/3F1 (against PEC13) and 357/3B6 (against PCS15) were generated. For further experiments, monoclonal antibodies were purified from culture supernatant by Protein G affinity chromatography.

### Polyclonal Antibodies

Polyclonal antibodies directed against PEC13 were generated according to standard procedures (see EP 1488209 A1, EP 1738178 A1). In brief, peptide PEC13 was coupled to the carrier protein KLH (Keyhole limpet hemocyanin) (PIERCE, Rockford, Ill., USA) using MBS (m-maleimidobenzoyl-N-hydroxysuccinimid Ester). With this conjugate sheep were immunized according to the following scheme: A sheep was initially immunized with 100 μg conjugate (mass refers to the peptide moiety of the conjugate) and boostered thereafter in four-weekly intervals with 50 μg conjugate each time. Four months after the initial immunization 300 ml antiserum were obtained from the sheep. Antigen-specific antibodies were purified from the antiserum as follows: 5 mg peptide PEC13 were coupled to 5 ml SulfoLink-gel (PIERCE, Rockford, Ill., USA). 50 ml antiserum were incubated with the gel batchwise for 4 hours at room temperature. The material was transferred in a column (empty NAP25 column, Pharmacia). The flow through was discarded, the gel was washed with 100 ml wash buffer (100 mM K-phosphate, 0.1% Tween 20, pH 6.8), and specifically bound antibodies were eluted with 50 mM citric acid, pH 2.7. The eluate was dialysed against 50 mM Na-phosphate, 100 mM NaCl, pH 8.0. The antibody yield was 36.6 mg.

### Labeling of Antibodies

Labeling was performed by standard procedures (see EP 1488209 A1, EP 1738178 A1): The concentration of the purified antibodies was adjusted to 1 g/L, and the antibodies were labelled by incubation with the chemiluminescent label MACN-Acridinium-NHS-Ester (1 g/L; InVent GmbH, Hennigsdorf, Germany) in a 1:5 molar ratio for 30 min at room temperature. The reactions were stopped by addition of 1/10 volume of 1 mol/L Tris for 10 min at room temperature. Labelled antibodies were separated from free label by size-exclusion chromatography on a NAP-5 column (GE Healthcare, Freiburg, Germany) and a Bio-Sil® SEC-400-5 HPLC column (BIO-RAD).

### Coating of Antibodies

Coating was done by standard procedures (see EP 1488209 A1, EP 1738178 A1): Polystyrene tubes (Greiner) were coated with purified antibodies (per tube, 2 μg of antibody in 300 μL of 10 mmol/L Tris, 10 mmol/L NaCl, pH 7.8) overnight at 22° C. Tubes were then blocked with 10 mmol/L Na-phosphate (pH 6.5) containing 3% Karion FP (Merck), 0.5% BSA protease free (Sigma) and lyophilized.

### Generation of a Standard Matrix

From a pool of sera from healthy human subjects PRX4 immunoreactivity was depleted by to affinity chromatography as follows: 5 mg antibody 340/3F1 were coupled to 5 ml CarboLink™ Coupling Gel (PIERCE, Rockford, Ill., USA), and the serum pool (total volume 200 ml; pool of 20 ml each of 10 individuals) was passed four times sequentially through the column

### Standards

Standards for immunoassays were prepared by making serial dilutions of either peptide PEE27 or an extract of total soluble proteins from porcine liver (SCIPAC, UK) in the standard matrix, depending on the type of assay (details are described later). Standards were stored at −30° C. until use. For the PEE27 standards, standard concentrations were assigned according to the weight of the peptide material. For the liver extract standards, arbitrary PRX4 concentrations were assigned [arb. U/l]. How many U correspond to which mass of immunoreactive PRX4 was roughly estimated by identifying the standard concentration, which is required to saturate the binding capacity of a tube coated with a defined amount of anti-PEC13 monoclonal antibody (2 μg/tube). Saturation was approximately achieved, when 50 μl of a standard was used, which had a PRX4 concentration of 300 arb. U/l.

### Immunoassays

Several sandwich immunoassays were set up using components described above.

A.1) Homologous Immunoassay with Monoclonal Antibodies/2-Step Version

The anti-PEC13 antibody 340/4F2 was used as solid phase antibody. The anti-PEC13 antibody 340/3F1 was used as labeled antibody. The assay buffer for the first incubation step was 300 mM K-phosphate, pH 7.0, 100 mM NaCl, 10 mM EDTA, 0.09% Na-azide, 0.5% BSA, 0.1% unspec. bovine IgG, 0.1% unspecific. sheep-IgG, 0.1% unspecific mouse IgG. Where indicated, DTT was added to the assay buffer at a concentration of 3 mM unless indicated differently. The assay buffer for the second incubation step was 300 mM K-phosphate, pH 7.0, 250 mM NaCl, 10 mM EDTA, 0.09% Na-azide, 0.5% BSA, 0.1% unspecific bovine IgG, 0.1% unspecific. sheep-IgG, 0.1% unspecific mouse IgG, and contained 10⁶ relative light units (RLU) of MACN-labeled antibody per 100 μl. In the first incubation step 100 μl standards or samples and 100 μl assay buffer were pipetted into the coated tubes. Tubes were incubated 20 hours at 22° C. under agitation. Then, the tubes were washed 4 times with 1 mL of B.R.A.H.M.S washing solution (B.R.A.H.M.S AG, Hennigsdorf, Germany). Then 100 μl of buffer containing the MACN-labeled antibody were added, and tubes were incubated 2 hours at 22° C. under agitation. Then, tubes were washed 4 times with 1 mL of B.R.A.H.M.S washing solution (B.R.A.H.M.S AG, Hennigsdorf, Germany), and bound chemiluminescence was measured for 1 s per tube with a LB952T luminometer (Berthold). Concentrations of samples were calculated using the Software MultiCalc (Spline Fit).

A.2) Homologous Immunoassay with Monoclonal Antibodies/1-Step Version

The same assay components as for assay A.1 were used. 100 μl standards or samples and 100 μl of buffer containing the MACN-labeled antibody were pipetted in the coated tubes, and tubes were incubated 2 hours at 22° C. under agitation. Then, tubes were washed 4 times with 1 mL of B.R.A.H.M.S washing solution (B.R.A.H.M.S AG, Hennigsdorf, Germany), and bound chemiluminescence was measured for 1 s per tube with a LB952T luminometer (Berthold). Concentrations of samples were calculated using the Software MultiCalc (Spline Fit).

B) Heterologous Immunoassay with Monoclonal Antibodies/2-Step Version

The anti-PCS15 antibody 357/3B6 was used as solid phase antibody. The anti-PEC13 antibody 340/4F2 was used as labeled antibody. An extract of soluble proteins from porcine liver was used as standard material (see above). All other conditions and procedures were as described for assay A.1.

C) Homologous Immunoassay with Polyclonal Antibodies/2-Step Version

Purified sheep anti-PEC13 antibody was used both as solid phase antibody and as labeled antibody. All other conditions and procedures were as described for assay A.1.

### Size-Exclusion Chromatography

A pool of human sera containing endogenous PRX4 immunoreactivity as well as an extract of soluble porcine liver proteins was fractionated using a Bio-Sil® SEC-400-5 HPLC column (BIO-RAD). The sample volume was 100 μl. The running buffer was 50 mM K-Phopshate, pH 7.4, 150 mM NaCl, 0.09% Na-Azide. The flow rate was 0.8 mL/min. 0.4 mL fractions were collected and PRX4 immunoreactivity was measured using assay A.1.

### Measurement of Clinical Samples

PRX4 was measured in serum samples from patients with various diseases. These were, cardiovascular diseases including chronic and acute heart failure, acute coronary syndrome, atherosclerosis, hypertension, stroke, transient ischemic attack (summarized as cardiac diseases), infectious diseases, sepsis, severe sepsis, septic shock (summarized as sepsis), pancreatitis, other diseases of the gastrointestinal tract including colitis ulcerosa, Morbus Crohn, cancer including colon, breast and pancreas cancer, diabetes mellitus, rheumatoid arthritis, chronic and acute kidney disease (summarized as kidney disease), Alzheimer's disease, mild cognitive disorders, Parkinson's disease (summarized as neurodegenerative disorders). Samples from healthy subjects were also measured.

### Results

### Antibody Design

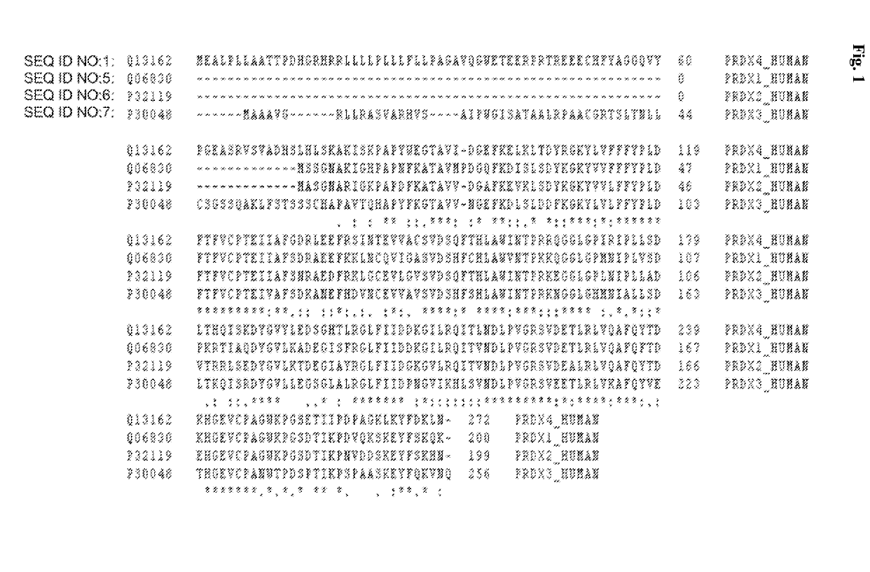

PRX4 belongs to a family of several related proteins. While nothing is known on the occurrence of these outside of tissue, we took this relationship into account in the design of the epitope specificity of the anti-PRX4 antibodies developed, and we synthesized peptides for immunization, which correspond to regions, which are located in the N-terminal part of PRX4, i.e. upstream of amino acid position 73. Such regions either do not exist in other members of the PRX protein family, or are lacking sequence homology with PRX4 (FIG. 1).

### Dose-Response Curves

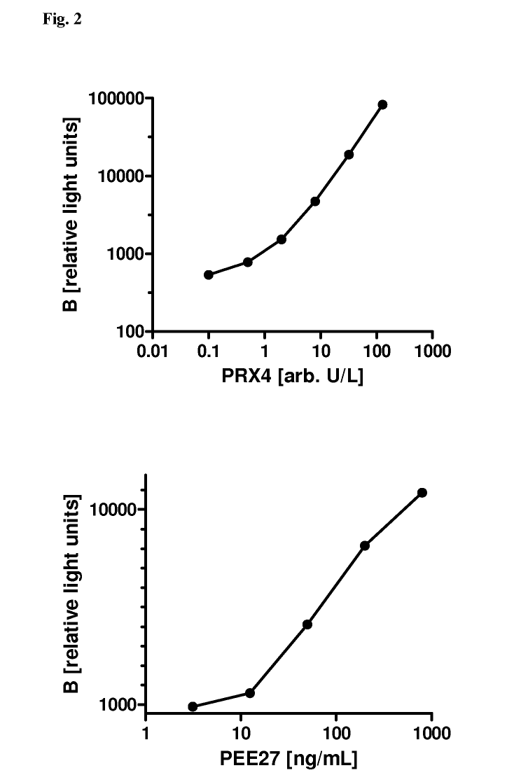

Using a homologous sandwich assay design for the measurement of PRX4 immunoreactivity (A.1), dose response curves could be created by employing either native analyte in form of a porcine liver extract or a synthetic peptide containing twice the epitope of the antibodies used (FIG. 2). Similar results were obtained using assay design C, i.e. when polyclonal antibodies instead of monoclonal antibodies were used (data not shown). With the assays PRX4 immunoreactivity could be detected in patient samples (described below).

### Nature of the Measured Analyte

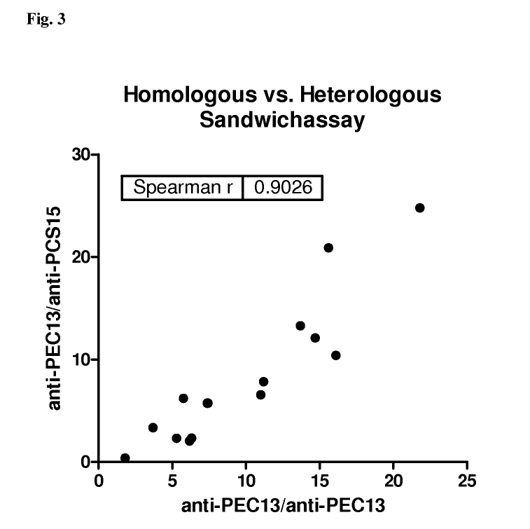

Homologous sandwich immunoassays for the measurement of multimeric analytes, i.e. sandwich assays, which utilize two antibodies with the same epitope specificity, might be more prone to cross-react non-specifically with other molecules than the intended target molecule, similar to competitive immunoassays, than heterologous sandwich immunoassays, which utilize two antibodies with different epitope specificities, a situation, which typically confers a very high analyte specificity. In order to assess whether or not the homologous PRX4 assay A.1 also detects other molecules in complex samples such as sera in addition to PRX4 immunoreactivity, patient samples containing PRX4 immunoreactivity were measured in the homologous assay A.1 and the heterologous assay B. The measured results for the two assays were highly correlated (r=0.9; FIG. 3), demonstrating that the homologous assay A.1 detects PRX4 immunoreactivity very specifically.

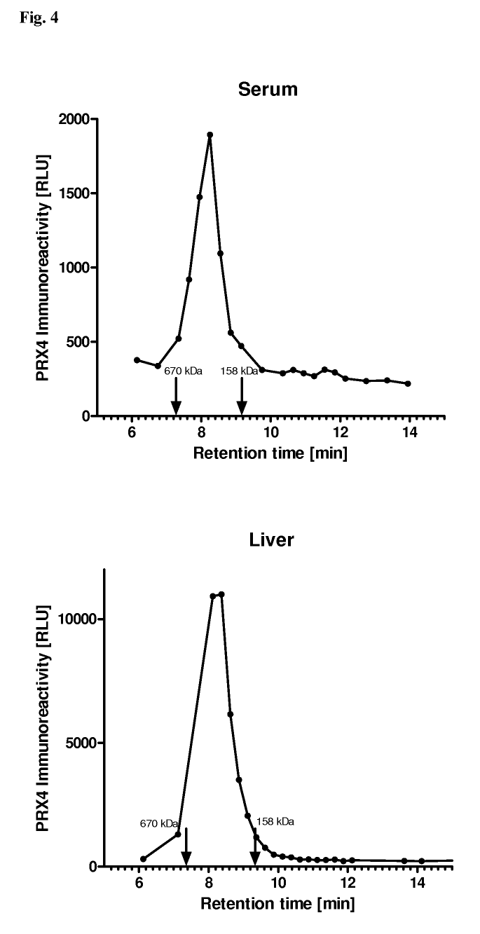

The apparent molecular weight of PRX4 immunoreactivity in neat serum (a pool of sera from sepsis patients was used) and liver extract was analyzed by size-exclusion chromatography followed by measurement of the resulting fractions using the assay A.1. In this analysis the PRX4 immunoreactivity detected in both neat serum and liver extract had an apparent molecular weight between 158 and 660 kDa, more specifically approximately 330 kDa (FIG. 4). This finding is compatible with the possibility that PRX4 might exist as a homomultimer, more specifically as a homodecamer or homopentamer, and/or as a heteromultimer, in which more than one molecule of PRX4 is associated with one or more protein of the same kind or different kinds (such as for instance PRX1).

### Effects of Reducing Agents

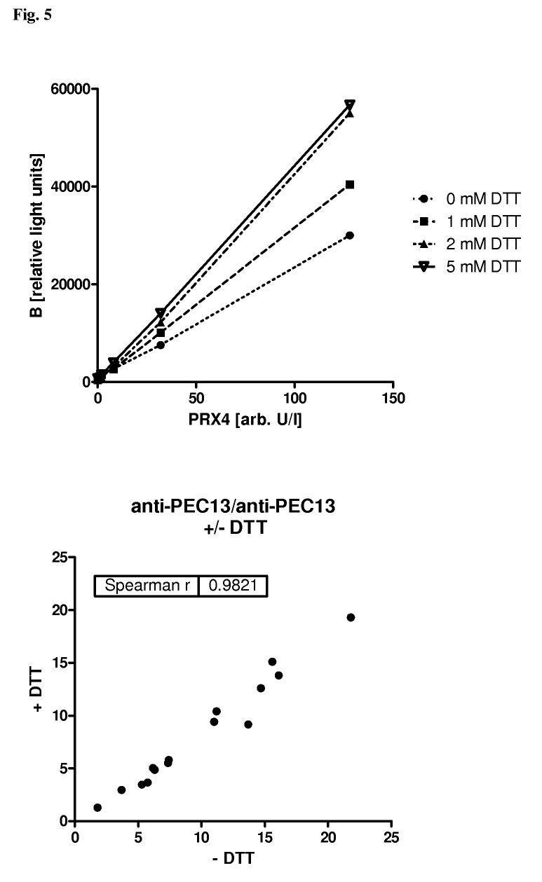

When a reducing agent such as DTT was added to the assay buffer used in the first incubation step of assay A.1, surprisingly the detected PRX4 immunoreactivity increased dramatically, both for porcine liver extract and human serum samples (FIG. 5). The effect was most pronounced at DTT concentrations above 2 mM and essentially plateaued between 2 and 5 mM. A suitable DTT concentration thus is 3 mM, which was used in further analyses. PRX4 immunoreactivity in human serum samples behaved as PRX4 from porcine liver extract, as demonstrated by an ideal correlation obtained, when samples were measured in the presence or absence of DTT (FIG. 5, Spearman r=0.98). The functional assay sensitivity of assay A.1 (+3 mM DTT), defined as the concentration at which the interassay CV was 20% was determined at 0.7 arb. U/l. The presumable mechanism responsible for the observed effect might be a partial reduction of disulfide bonds within PRX4 multimers leading to smaller PRX4 multimers and concomitantly exposure of previously inaccessible epitopes.

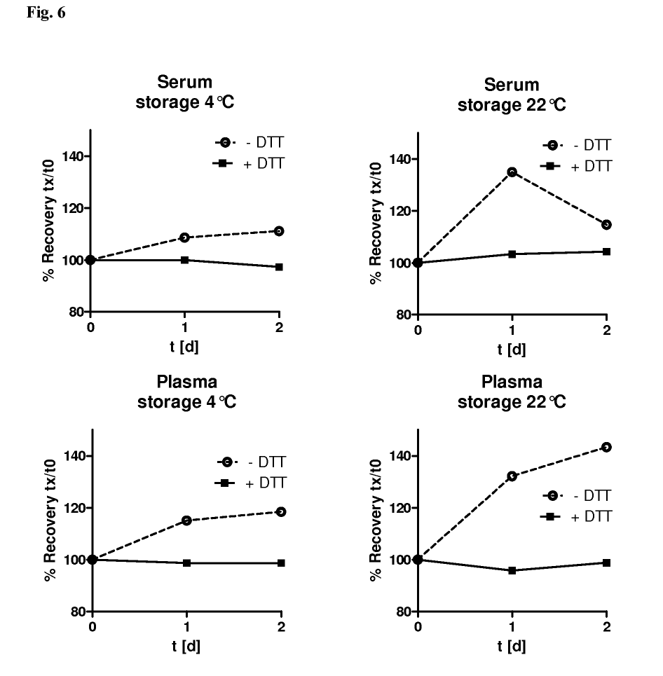

An additional effect observed by the addition of DTT to the assay buffer was an improvement of the apparent ex vivo stability of PRX4 immunoreactivity: When DTT was omitted from the assay buffer used in the first incubation step of assay A.1, the detectable PRX4 immunoreactivity in human sera or plasma increased as a function of ex vivo storage time of the samples (FIG. 6), the increase being more pronounced at 22° C. than at 4° C. Surprisingly, addition of 3 mM DTT to the assay buffer used in the first incubation step of assay A.1 prevented this apparent increase of PRX4 immunoreactivity. It can be speculated that upon storage of samples endogenous PRX4 multimers undergo structural changes (potentially mediated by proteases, or other effectors) leading to the partial exposure of otherwise inaccessible or less accessible epitopes. The structural change of PRX4 multimers mediated by the addition of DTT then leads to a much stronger increase of the PRX4 immunoreactivity than the increase induced by sample storage, making it finally irrelevant, whether a sample has been stored or not, as long as the sample is contacted with a reducing agent such as DTT.

The observed beneficial effects of reducing agents such DTT can be obtained by contacting the sample at any stage, i.e. the reducing agent can be added to the sample directly prior the assay, or it can be included in the assay incubation with the sample.

Analogous to the beneficial effects of reducing agents such DTT as observed in the homologous assay (assay A), the same beneficial effects of reducing agents such DTT concerning analytical sensitivity and ex vivo stability were observed also, when a heterologous sandwich assay for detecting PRX4 immunoreactivity was used (assay B). As in assay A, measured concentrations of PRX4 in human serum samples were unaffected also in assay B by the addition of DTT (when samples were measured with assay B in the absence or presence of 3 mM DTT in the assay buffer used in the first incubation step of assay; measured values for presence or absence of DTT strongly correlated (Spearman r=0.96)). The correlation was also strong (Spearman r=0.85), when the heterologous assay B was compared with the homologous assay A.1, both run in the presence of DTT.

### Measurement of Clinical Samples

Since it has been suggested that PRX4 might be expressed in blood cells, we assessed the effect of hemolysis on the measured immunoreactivity in plasma samples. It was observed that PRX4 immunoreactivity could be detected only when severe hemolysis occurred, i.e. when the sample was visibly red (hemoglobin concentration of 400 g/dL and above). Samples with such degree of hemolysis are typically withheld from any type of analysis in the routine laboratory. Thus, the potential negative effect of hemolysis on the accuracy of the PRX4 determination is practically not important.

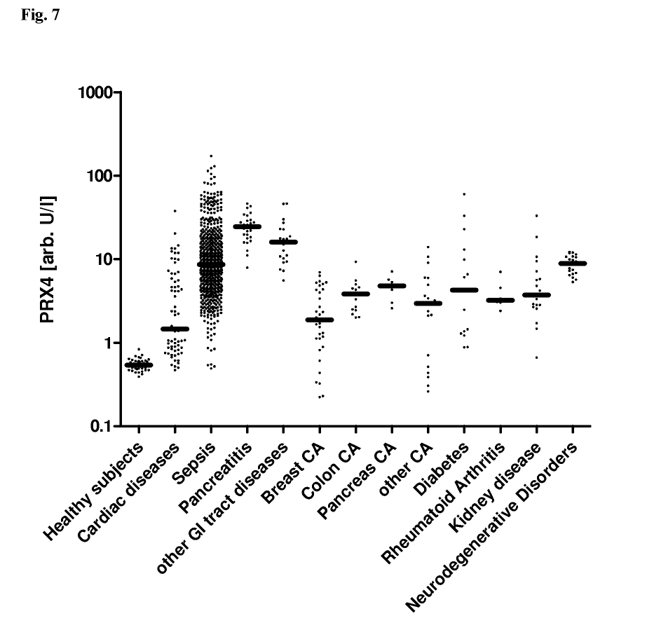

PRX4 was measured in serum samples from patients with various diseases (FIG. 7). These were, cardiovascular diseases including chronic and acute heart failure, acute coronary syndrome, atherosclerosis, hypertension, stroke, transient ischemic attack (summarized as cardiac diseases), infectious diseases, sepsis, severe sepsis, septic shock (summarized as to sepsis), pancreatitis, other diseases of the gastrointestinal tract including colitis ulcerosa, Morbus Crohn, cancer including colon, breast and pancreas cancer, diabetes mellitus, rheumatoid arthritis, chronic and acute kidney disease (summarized as kidney disease), Alzheimer's disease, mild cognitive disorders, Parkinson's disease (summarized as neurodegenerative disorders). Samples from healthy subjects were also measured. Levels of PRX4 from healthy subjects were mostly above the functional sensitivity of the assay, but were the lowest compared with all other groups of patients investigated. The highest levels were detected in patients with sepsis, pancreatitis and other diseases of the gastrointestinal tract. Since elevation of PRX4 is observed in many types of diseases, the potential of a diagnostic use of PRX4 measurement appears low at first glance. However, this most likely is an underestimation, because not all these patients are presenting with the same symptoms and clinical conditions, and the value of any laboratory measurement in general must be seen in the context of the symptoms and conditions. PRX4 as an enzyme involved in the regulation of oxidative stress must be assumed to reflect the level of oxidative stress, and thus a measure for the acuteness and/or severity of the disease process.

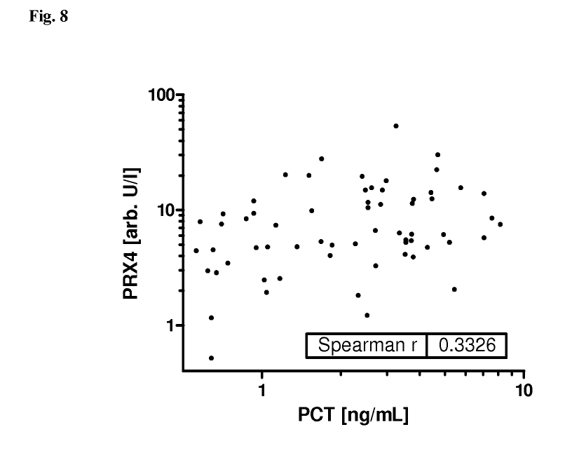

In patients with sepsis, severe sepsis and septic shock, PRX4 was correlated with PCT (Spearman r=0.33) (see FIG. 8). In such patient population increasing PCT levels are known to be associated with increasing severity of the disease Miller B, et al. (2000) Crit Care Med. 28(4):977-831.

### Example 2

### Comparison of Plasma and Serum Samples

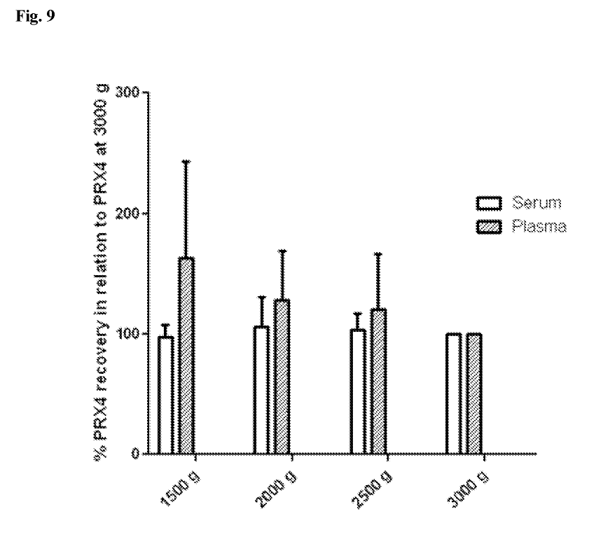

Serum and EDTA plasma was obtained from ten individuals in several aliquots. The aliquots were centrifuged at different centrifugation forces (at 1500, 2000, 2500 and 3000 g, respectively) for 15 minutes to separate serum and plasma, respectively, from solid blood compounds (blood cells etc.). PRX4 was measured in the sera and plasmas, and PRX4 concentrations obtained for 1500, 2000, 2500 g for each individual and sample matrix were divided by the respective value obtained after centrifugation at 3000 g (only values above the functional assay sensitivity were included). The results are shown in FIG. 9. Shown are means and standard deviations for those calculated values. The experiment illustrated in FIG. 9 demonstrates that serum values do not depend on the centrifugation force applied, whereas mean plasma values increase with decreasing centrifugation force. Additionally, the precision for plasma values is much worse than for serum values.

In conclusion, plasma as opposed to serum contains non-soluble PRX4 immunoreactivity, and extreme centrifugation conditions are required to remove these. If centrifugation is performed insufficiently, then detection of falsely elevated PRX4 values with bad precision can result.

TABLE 1
Mean coefficient of variation (CV) for serum and plasma samples

| | |
|---|---|
| Serum | Plasma |

| | <FAS | >FAS | <FAS | >FAS |
|---|---|---|---|---|
| n = | 36 | 114 | 11 | 139 |
| mean CV | ./. | 4.0% | ./. | 5.8% |

Serum and plasma samples were also obtained in parallel from 150 healthy individuals by centrifuging the blood monovettes as follows: serum: 15 min, 2000 g; plasma 15 min 3000 g. PRX4 was measured in duplicates. For plasma, more samples (n=139) gave values above the functional assay sensitivity (FAS) than for serum (n=114). The mean of the coefficients of variation (CVs) from the duplicate measurement from all values above the FAS was calculated, see Table 1. For the plasma samples, the CV was considerably higher (5.8%) than for the serum samples (4.0%).

In conclusion, even when a strong centrifugation force (e.g. 3000 g) is applied for obtaining plasma, the precision of PRX4 values is worse than for corresponding serum samples. Additionally, the finding that more plasma samples than serum samples are giving PRX4 values above the FAS, indicates that even under strong centrifugation conditions, more falsely elevated PRX4 values can potentially be obtained in plasma than in serum.

### Example 3

PRX4 was measured in patients with sepsis on consecutive days after admission.

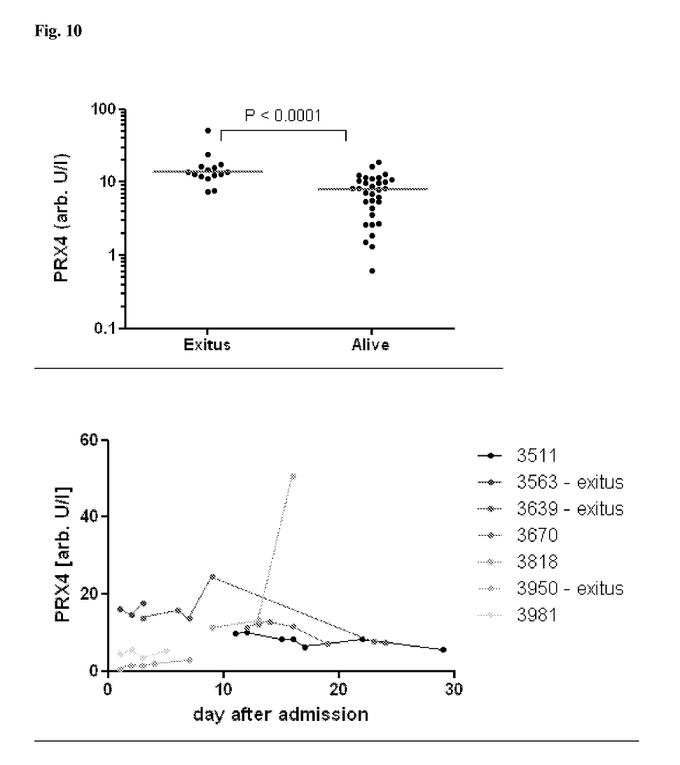

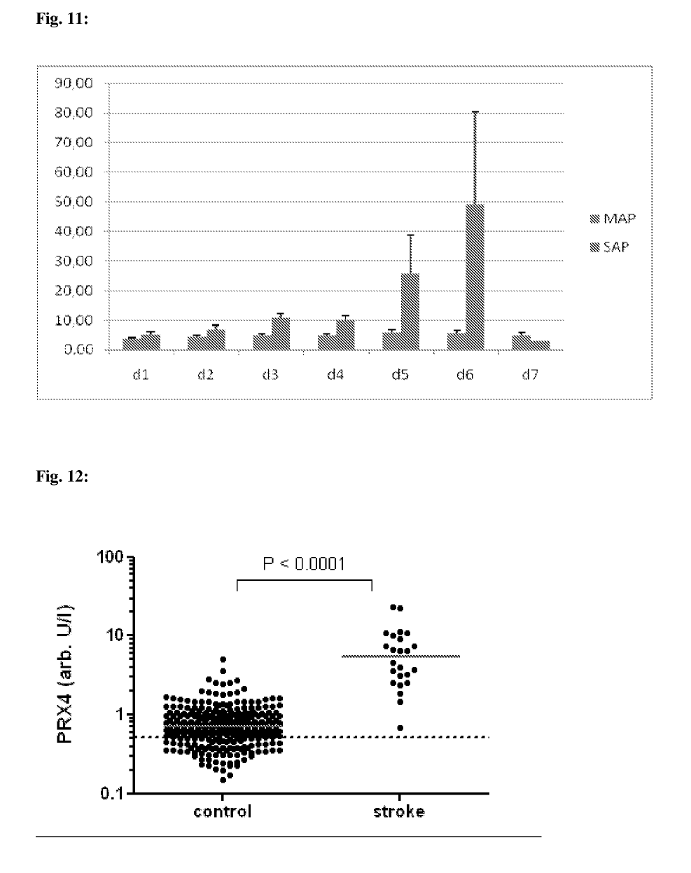

FIG. 10 shows the PRX4 values of 16 patients with sepsis (median 9.9 arb. U/l; range, 0.62-50.7 arb. U/l)

There was a significant difference (P<0.0001) between mean PRX4 values of all values from patients that died and patients that survived (16.2 vs 7.7 arb. U/l, respectively).

Patients who died exhibited a higher PRX4 value than survivors.

### Example 4

Samples of patients with pancreatitis were measured. PRX4-concentrations of 368 patients with pancreatitis were between 0.378 arb. U/l and 80.6 arb. U/l, median 4.53 arb.U/l.

Patients with severe acute pancreatitis (SAP) exhibited at least on day 3 after appearance of symptoms higher PRX4-values than patients with mild acute pancreatitis (MAP).

### Example 5

PRX4 values in samples were measured in patients with stroke.

Samples of 24 patients with stroke were measured [median 5.5 arb. U/l; range, 6.674-22.9 arb. U/l]. Patients with stroke exhibited higher PRX4 values than subjects of the control group.

1.-18. (canceled)

19. A method for the diagnosis or differential diagnosis or prognosis of a disease or clinical condition in a subject or for risk stratification or therapy monitoring or therapy guidance in a subject comprising the steps of:
(i) providing a sample of bodily fluid of a subject,
(ii) determining the level of peroxiredoxin 4 (PRX4) or a fragment thereof having at least 20 amino acids residues in length in said sample, and
(iii) correlating the level of PRX4 or a fragment thereof with a disease or clinical condition.

20. The method according to claim 19 with the proviso that the disease or clinical condition is not a disease or clinical condition selected from the group consisting of rheumatoid arthritis, osteoarthritis and ankylosing spondylitis.

21. The method according to claim 19, wherein the disease or clinical condition is associated with oxidative stress.

22. The method according to claim 19, wherein the disease or clinical condition is selected from the group consisting of infectious disease, cardiac disease, sepsis, pancreatitis, diseases of the gastrointestinal tract, cancer, diabetes mellitus, rheumatoid arthritis, kidney disease, and neurodegenerative disorders.

23. The method according to claim 22, wherein the disease or clinical condition is selected from the group consisting of infectious disease, cardiac disease, sepsis, pancreatitis, diseases of the gastrointestinal tract, cancer, diabetes mellitus, kidney disease, and neurodegenerative disorders.

24. The method according to claim 19, wherein the level of PRX4 or a fragment thereof in the sample is determined by contacting the sample with at least one PRX4 binder.

25. The method according to claim 24, wherein the at least one binder is an antibody, preferably a monoclonal antibody.

26. The method according to claim 19 wherein PRX4 or a fragment thereof is determined in a sandwich immuno assay.

27. The method according to claim 19, wherein prior to or during the determination of PRX4 or the fragment thereof the sample is contacted with a reducing agent such as dithiothreitol (DTT).

28. The method according to claim 19, wherein the sample of bodily fluid is selected from the group consisting of a blood sample, a serum sample, a plasma sample, a cerebrospinal fluid sample, a saliva sample, a solubilised tissue sample and an urine sample or an extract of any of the aforementioned samples.

29. The method according to claim 28, wherein the sample is a serum sample.

30. The method according to claim 19, wherein in addition a clinical parameter selected from the group consisting of age, gender, systolic blood pressure, diastolic blood pressure, antihypertensive treatment, body mass index, presence of diabetes mellitus and current smoking and/or a further laboratory parameter is determined.

31. The method according to claim 19, wherein an antibody is used that binds to an epitope contained in positions 1 to 73 of PRX4 according to SEQ ID NO:1 and is less than 20% cross-reactive with PRX4-related proteins.

32. An antibody that binds to an epitope contained in positions 1 to 73 of PRX4 according to SEQ ID NO:1 and is less than 20% cross-reactive with PRX4-related proteins.

33. The antibody according to claim 32, wherein the antibody binds to an epitope contained in positions 39-65 of PRX4 according to SEQ ID NO:1.

34. A kit comprising at least one antibody according to claim 32.

35. A method of claim 19 wherein an organ- or tissue-extract and/or enriched or purified fractions thereof containing PRX4 and/or a fragment thereof having at least 20 amino acids residues in length is used as a source for providing calibrators and/or control samples in the step of determining the level of PRX4 and/or a fragment thereof having at least 20 amino acids residues in length in a sample.

36. A method for diagnosing, prognosticating or monitoring a diseases or clinical condition which is an infectious disease, cardiac disease, sepsis, pancreatitis, diseases of the gastrointestinal tract, cancer, diabetes mellitus, rheumatoid arthritis, kidney disease, or neurodegenerative disorder which comprises using a method according to claim 19.
